(19) United States
(12) Patent Application Publication
(10) Pub. No.: US 2023/0310680 A1
(43) Pub. Date: Oct. 5, 2023

(54) FLUID CONTAINMENT LAYER AND PAD CONTAINING FLUID CONTAINMENT LAYER

(71) Applicant: Zephyros, Inc., Romeo, MI (US)

(72) Inventors: Christophe Chaut, Molsheim (FR); Greg Thompson, Simpsonville, SC (US); Karsten Scholz, Kriftel (DE); Marc Engel, Lingolsheim (FR)

(21) Appl. No.: 18/022,845

(22) PCT Filed: Oct. 21, 2021
(86) PCT No.: PCT/US2021/055947

§ 371 Date: Feb. 23, 2023

Related U.S. Application Data
(60) Provisional application No. 63/104,833, filed on Oct. 23, 2020.

(51) Int. Cl. A61L 2/18; B32B 5/02; B32B 5/26; B32B 5/12

(52) U.S. Cl. CPC A61L 2/18; B32B 5/02; B32B 5/26; B32B 5/12; A61M 35/00

(57) ABSTRACT

A fluid containment layer adapted to be used in a pad for absorption, containment, and/or controlled release of liquid or gel, where the fluid containment layer absorbs and/or holds a liquid or gel, such as a disinfecting liquid or gel. The fluid containment layer is resilient and returns to its original shape upon pressure applied thereto. The fluid containment layer includes generally vertically oriented fibers. The fluid containment layer may be used in a self-disinfecting pad.

### CLAIM OF BENEFIT OF FILING DATE

This application claims the benefit of U.S. Provisional Application No. 63/104,833, filed Oct. 23, 2020, the contents of which are hereby incorporated by reference in their entireties.

### FIELD

The present teachings generally related to a material for retaining a liquid or gel, and more particularly, to a material for absorbing, retaining, and/or controllably releasing a liquid or gel.

### BACKGROUND

Touchpoints in public areas are particularly concerning for the spread of bacteria, viruses, and fungi. Germs can last on various surfaces, especially hard surfaces, for hours or even days. Additionally, in settings where there are people who are known to be ill, such as hospitals, doctors' offices, or clinics, there is an even higher chance of coming into contact with pathogens on surfaces. During flu season or periods of rapid spread of highly contagious diseases, people are especially vigilant in minimizing contact with commonly touched surfaces, though sometimes contact with these surfaces is unavoidable.

For example, exiting a room or a building typically requires contact with a door. Even if the door is opened simply by pushing the door or a button, part of one's body or an object connected to or held by the person must contact the door or button to cause the door to open. These surfaces are repeatedly touched before they are able to be cleaned and sanitized.

Existing products provide self-disinfecting door push pads and contact surfaces, such as those described in U.S. Publication No. 2018/0169281, detailing devices that contain liquid or gel-like materials within a storage medium layer. However, these materials still have challenges and disadvantages. The process of getting the gel into the storage medium layer can be challenging, expensive, and time-consuming. The storage medium layer may be prone to sagging as a result of the weight of the liquid or gel-like material. The storage medium layer may not be able to hold enough liquid or gel-like material to last an extended period of time, or the liquid or gel-like material may dry up. As such, the device has to be discarded after using for a matter of days (e.g., a week or less). This also creates waste.

Therefore, there is a need for an improved disinfection pad, or layers thereof. There is a need for an improved fluid containment layer for any application requiring absorbing fluid (e.g., liquid or gel), holding fluid, controllably releasing fluid, or any combination thereof (e.g., in applications not necessarily limited to disinfection pads). There is a need for faster processing of the fluid containment layer when filling the fluid containment layer (e.g., by impregnating the material with gel). There is a need for a fluid containment layer that is capable of retaining a greater amount of fluid, such as a liquid, a gel, and/or a disinfecting material. There is a need for a fluid containment layer that does not sag. There is a need for a fluid containment layer that is capable of being refilled to reduce waste.

### SUMMARY

The present teachings meet one or more of the above needs by the improved devices and methods described herein. The present teachings include a material that is capable of containing a liquid or gel. The present teachings include a material that is capable of controllably releasing a liquid or gel. The present teachings include a material that is resistant to sag or that sags less than a traditional pad material with the same amount of liquid or gel. The present teachings include a material that holds a greater amount of liquid or gel than a traditional pad material, such as that described in U.S. Publication No. 2018/0169281. The present teachings include a material that aids in distribution of liquid or gel throughout the material and through adjacent layers to a user contacting the outermost layer of the disinfection pad. The present teachings include a material that provides a resilient material able to withstand repeated contact by a user.

The present teachings include a fluid containment layer that absorbs and retains a liquid or gel. The fluid containment layer may be used in a pad for controlled release of the liquid or gel. The fluid containment layer may be used in a self-disinfecting pad. The liquid or gel may be a disinfecting liquid or gel. Liquid or gel is released upon a user exerting pressure on the pad containing the fluid containment layer. The fluid containment layer may have generally vertically oriented fibers. The vertically oriented fibers may be generally vertically oriented in an uncompressed state or prior to undergoing any compression operations. The fibers may be oriented generally vertically (e.g., vertical in the thickness direction) as a result of a vertical lapping process or air laying process, for example. The fluid containment layer may be resilient such that it returns to its original shape upon release of pressure applied thereto. The fluid containment layer may maintain a generally even distribution of liquid or gel within the fluid containment layer. Upon release of the liquid or gel from the fluid containment layer at one or more points, remaining liquid or gel within the fluid containment layer may disperse to maintain the generally even distribution. The fluid containment layer may have a gradient density structure, wherein through the thickness, the density increases from one surface to an opposing surface. The fluid containment layer may have different portions that absorb or hold different amounts of fluid. The different portions may have different saturation points. The gradient structure may be in the thickness direction. The fluid containment layer may have a single layer having a gradient structure therein. The gradient structure may be formed from two or more layers creating the fluid containment layer.

The fluid containment layer may allow for movement of the liquid or gel in multiple directions within the fluid containment layer to distribute the liquid or gel. The fluid containment layer may be resistant to sag at its saturation point. The fluid containment layer may comprise superabsorbent fibers. The fluid containment layer may be impregnated with a powder so that upon addition of a liquid (e.g., a liquid containing alcohol), a gel is created and held within the fluid containment layer. The fluid containment layer may be generally planar. The fluid containment layer may be shaped into a three-dimensional structure. The fluid containment layer may be thermoformable. The fluid containment layer may be moldable. The fluid containment layer may be refillable.

The present teachings also contemplate a self-disinfection pad including the fluid containment layer as described herein.

### BRIEF DESCRIPTION OF THE DRAWINGS

FIG. 1 is a cutaway view of an exemplary disinfection pad in accordance with the present teachings.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the description herein, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

Disinfection pads exist to provide a self-disinfecting surface for a user to contact, thereby reducing the spread of germs. The disinfection pad is positioned or installed in or on high-touch areas, such as doors. When a user touches the disinfection pad, disinfecting or antibacterial liquid or gel is released at the touchpoint to disinfect the area contacted, the part of the body the person used to touch the surface, or both. For example, the disinfection pad could be positioned on a door. A person could contact the disinfection pad with his or her hand to open the door. The area the person touches is disinfected as a result of the pressure from the person's hand on the disinfection pad releasing an amount of disinfecting or antibacterial substance. The person's hand may also be disinfected.

A pad that absorbs, retains, controls release of fluids, or any combination thereof, may include multiple layers or components. For example, the pad may be a disinfection pad. While referred to herein as a disinfection pad, it is contemplated that the same or similar features may be present in a pad that does not necessarily have disinfection capabilities or uses. For example, the pad may be used to moisten another article or surface. The pad may be used to controllably release some type of fluid (e.g., gel or liquid), that does not necessarily provide disinfection. Any of these other uses are considered to be within the scope of the present teachings. The disinfection pad may include a backing layer. The disinfection pad may include a fluid containment layer. The disinfection pad may include an intermediate layer. The disinfection pad may include a contact layer. The disinfection pad may include additional layers. Additional layers may include layers providing structural properties, layers providing fluid transfer or distribution properties, layers having adhesives or other attachment functions (e.g., attachment of one layer to another or attachment of the disinfection pad to a surface), or the like.

The present teachings also relate to a material suitable for containing a liquid or a gel. For example, the material may act as a fluid containment layer for a disinfection pad. While referred to herein as a fluid containment layer, or referring to a fluid within the layer, it is understood that “fluid” herein may be liquid, gel, or both. The fluid or fluid containment layer may include or contain solid particles distributed throughout the liquid or gel. The fluid or fluid containment layer may contain solid particles that become a gel upon introduction of a liquid.

The disinfection pad may include a backing layer. The backing layer may act to contain one or more other layers of the disinfection pad. For example, the backing layer may act as a tray. The backing layer may have one or more side walls to define an area for containing one or more of the layers. The backing layer may act to at least partially prevent or reduce air flow or exposure of the fluid containment layer to air flow. The backing layer may reduce or prevent drying out of the fluid containment layer. The backing layer may be generally non-permeable to prevent or reduce undesired leakage of the fluid within the disinfection pad. The backing layer may have one or more channels, grooves, reservoirs, or the like (e.g., at least on the internal side of the backing layer facing the fluid containment layer), for containing additional fluid. The additional fluid may then be absorbed by the fluid containment layer as fluid is dispensed or used. The backing layer may be flexible. The backing layer may be rigid.

The outer side of the backing layer (e.g., the side opposite the internal side that faces the fluid containment layer) may be adapted to contact or be secured to a surface or another layer of the disinfection pad. The backing layer may be molded into a desired shape. The backing layer may, for example, have a generally planar outer side or portion of the outer side adapted to contact a generally planar surface. As an example, the backing layer may be secured to a high-contact surface, such as an area frequently contacted for opening or closing a door. The backing layer, or at least a portion thereof, may have a generally complementary shape to the area upon which it is to be secured or contact. For example, if the backing layer is to be secured around a handle (e.g., a doorknob, door handle, shopping cart or pushcart handle or bar), the backing layer may have a shape generally complementary to the handle so that the backing layer can be secured thereto without rocking, sliding, or otherwise moving from the area upon which it is to be secured.

The outer perimeter of the backing layer may be a shape that generally matches the shape of the area to which the disinfection pad is to be secured. For example, a disinfection pad may be located on a button (e.g., elevator button, touchpad, handicap door pushbutton). The outer perimeter of the backing layer may be generally the same shape as that button or smaller. The outer perimeter of the backing layer may be generally the same size and/or shape as a push plate for a door.

The fluid containment layer may function to hold, contain, and/or release a liquid or gel. The fluid containment layer may function to hold or contain an antibacterial, antimicrobial, and/or disinfecting fluid (e.g., a liquid or gel). The fluid containment layer may provide resilience, compression resistance, moisture transference (e.g., fluid is moved into, through, and/or across the fluid containment layer, into an adjacent layer, or a combination), or a combination thereof. The fluid containment layer may be shaped to fit the area to which it will be installed. For example, the fluid containment layer may be shaped to fit within the confines of the backing layer. The fluid containment layer may have one or more contours (e.g., as viewed from a cross-section of the thickness of the material). The contours may match the contours of the backing layer, surface upon which the disinfection pad is to be positioned, or both. The fluid containment layer may be lightweight, washable, reusable, or a combination thereof.

The fluid containment layer may include one or more layers of fibrous material. The fluid containment layer may include one or more fibrous layers having fibers arranged in a generally vertical orientation (e.g., vertical in the thickness direction).

The fluid containment layer may transfer fluid from and/or to one or more abutting layers. The fluid containment layer may absorb fluid from the backing layer (e.g., fluid collected in the backing layer and/or one or more channels or reservoirs).

The fluid containment layer may have a high loft (or thickness) at least in part due to the orientation of the fibers (e.g., oriented generally transverse to the longitudinal axis of the layer) of the layer and/or the methods of forming the layer. The fluid containment layer may exhibit good resilience and/or compression resistance. The fluid containment layer may be resistant to puncturing. The fluid containment layer, due to factors such as, but not limited to, unique fibers, surfaces, physical modifications to the three-dimensional structure (e.g., via processing), orientation of fibers, or a combination thereof, may exhibit good moisture transfer and/or absorption characteristics versus traditional materials. The fluid containment layer may exhibit good fluid retention. The fluid containment layer may be resistant to sagging, even when fully saturated with fluid. The fluid containment layer may have a higher saturation point than traditional disinfection pads, where saturation point is the maximum volume of fluid capable of being absorbed within the fluid containment layer.

The fluid containment layer may be adjusted based on the desired properties. The fluid containment layer may be tuned to provide a desired weight, thickness, compression resistance, or other physical attributes. The fluid containment layer may be tuned to provide a desired moisture absorption or moisture transfer rate. The fluid containment layer may be tuned to provide a desired drying rate. The drying rate may be very slow to retain the disinfection fluid within the material and extend the life of the disinfection pad. The fluid containment layer may be formed from nonwoven fibers. The fluid containment layer may be a nonwoven structure. The fluid containment layer may be a lofted material. The fluid containment layer may be thermoformable so that the layers may be molded or otherwise manufactured into a desired shape to meet one or more application requirements.

The fluid containment layer may have a generally uniform distribution of fibers. The fluid containment layer may have a generally uniform density throughout the thickness of the material. The fluid containment layer may have a varying structure through the thickness.

The fluid containment layer may have a gradient structure where the density of the material changes from one portion of the fluid containment layer to another. The material may gradually become more rigid. For example, the fluid containment layer may have a softer surface at one end or one surface, and a harder surface at an opposing end or opposing surface. The gradient structure may be in the thickness direction. The gradient structure may be across the length or the width of the material. The gradient structure may further enhance the fluid transfer rate from one side or surface to another.

The fluid containment layer may have a gradient structure where different portions of the fluid containment layer absorb or hold different amounts of fluid. Different portions or areas of the fluid containment layer may have different saturation points. For example, the fluid containment structure may have a gradient structure in the thickness direction. Toward one surface of the fluid containment structure, a greater volume of fluid may be absorbed and/or held within the material than the volume of fluid absorbed and/or held toward an opposing surface of the fluid containment structure. The gradient structure may occur within a single layer of material (e.g., as a result of fibers or other fillers used, the density of the material, processing techniques, the like, or a combination thereof). The gradient structure may occur through two or more layers arranged in generally planar contact to form the fluid containment layer.

As an example, the fluid containment layer may have a surface facing the backing layer and a surface facing the contact layer, with the thickness direction being the direction extending between the surfaces. Through the thickness of the fluid containment layer, the liquid or gel may be held toward the surface facing the backing layer. Approaching the surface facing the contact layer, less volume of liquid or gel may be present. This may allow for further control of the amount of fluid released upon a user upon contact with the contact layer. Positioning a greater volume of fluid further from the contact layer may also control or delay the evaporation or drying out of the pad since a higher volume of fluid is located further away from any air exposure (e.g., air exposure as a result of the openings in the contact layer and/or upon contact with the contact layer).

The fluid containment layer may have pores. The pores may be formed from interstitial spaces between the fibers and/or the shape (e.g., by having a multi-lobal or deep-grooved cross-sectional fiber) of the fibers. The pores may extend throughout the entire thickness of the fluid containment layer. The pores may extend through a portion of the thickness of the fluid containment layer. The pores and/or the vertical orientation of the fibers may create a capillary effect or chimney effect for absorbing, retaining, distributing, and/or removing fluid from one surface or portion of the fluid containment layer and transferring to another area (e.g., to an opposing surface of the fluid containment layer, to an intermediate layer). For example, the fluid containment layer may push and/or pull the fluid from a first surface of the fluid containment layer to an opposing second surface of the fluid containment layer through the thickness of the fluid containment layer. Capillary effect, or capillary action, is the ascension of liquids through a tube, pore, cylinder, or permeable substance due to adhesive and cohesive forces interacting between the liquid and the surface. The diameter of the pores or channels defined by the fibers (e.g., forming a capillary) for movement of liquid may be selected based on the thickness of the material through which the liquid must travel. A thinner diameter capillary or channel may see the liquid rise higher than liquid in a larger diameter capillary or channel due to capillary action because of adhesive forces.

Distribution of the fluid may not be limited to travel in the vertical direction through the thickness of the material. It is further contemplated that the fluid may be able to disperse or distribute through the material in the length direction, in the width direction, or on an angle from the vertical direction. Fluid may travel along the surfaces of the fibers. The fluid may travel to an area where there is less fluid to maintain an even distribution of fluid within the fluid containment layer. For example, when a user applies pressure to the disinfection pad, causing fluid to rise to the outermost layer of the disinfection pad, fluid is necessarily removed from the fluid containment layer at that touchpoint. Fluid within the fluid containment layer may then travel to the area where fluid has been removed to maintain a generally even distribution of fluid within the fluid containment layer.

The ability of the fluid containment layer to pull or push moisture through the layer, across the layer, or at an angle may be, at least in part, due to the geometries of the fibers and/or the voids between the fibers (e.g., creating channels or pores). The fibers may have a cross-section that is substantially circular or rounded. The fibers may have a cross-section that has one or more curved portions. The fibers may have a cross-section that is generally oval or elliptical. The fibers may have a cross-section that is non-circular. Such non-circular cross-sections may create additional tubes or capillaries within which the moisture can be transferred. For example, the fibers may have geometries with a multi-lobal cross-section (e.g., having 3 lobes or more, having 4 lobes or more, or having 10 lobes or more). The fibers may have a cross-section with deep grooves. The fibers may have a substantially “Y”-shaped cross-section. The fibers may have a polygonal cross-section (e.g., triangular, square, rectangular, hexagonal, and the like). The fibers may have a star shaped cross-section. The fibers may be serrated. The fibers may have one or more branched structures extending therefrom. The fibers may be fibrillated. The fibers may have a cross-section that is a nonuniform shape, kidney bean shape, dog bone shape, freeform shape, organic shape, amorphous shape, or a combination thereof. The fibers may be substantially straight or linear, hooked, bent, irregularly shaped (e.g., no uniform shape), or a combination thereof. The fibers may include one or more voids extending through a length or thickness of the fibers. The fibers may have a substantially hollow shape. The fibers may be generally solid. The shape of the fibers may define capillaries or channels through which moisture can travel (e.g., from one side of the fluid containment layer to an opposing side of the fluid containment layer).

The fibers that make up the fluid containment layer (or any other layer of the disinfection pad) may have an average linear mass density of about 0.5 denier or greater, about 1 denier or greater, or about 5 denier or greater. The material fibers that make up the fluid containment layer may have an average linear mass density of about 25 denier or less, about 20 denier or less, or about 15 denier or less. Fibers may be chosen based on considerations such as cost, resiliency, desired moisture absorption/resistance, or the like. For example, a coarser blend of fibers (e.g., a blend of fibers having an average denier of about 12 denier) may help provide resiliency to the fluid containment layer. A finer blend (e.g., having a denier of about 10 denier or less or about 5 denier or less) may be used, for example, if a softer material is required. The fibers may have a staple length of about 1.5 millimeters or greater, or even about 70 millimeters or greater (e.g., for carded fibrous webs). For example, the length of the fibers may be between about 30 millimeters and about 65 millimeters. The fibers may have an average or common length of about 50 to 60 millimeters staple length, or any length typical of those used in fiber carding processes. Short fibers may be used (e.g., alone or in combination with other fibers) in any nonwoven processes. For example, some or all of the fibers may be a powder-like consistency (e.g., with a fiber length of about 3 millimeters or less, about 2 millimeters or less, or even smaller, such as about 200 microns or greater or about 500 microns or greater). Fibers of differing lengths may be combined to provide desired properties. The fiber length may vary depending on the application; the moisture properties desired; the type, dimensions and/or properties of the fluid containment layer (e.g., density, porosity, desired air flow resistance, thickness, size, shape, and the like of the fluid containment layer and/or any other layers of the disinfection pad); or any combination thereof. The addition of shorter fibers, alone or in combination with longer fibers, may provide for more effective packing of the fibers, which may allow pore size to be more readily controlled in order to achieve desirable characteristics (e.g., moisture interaction characteristics).

The fluid containment layer (or any other layer of the disinfection pad) may include fibers blended with the inorganic fibers. The fluid containment layer may include natural, manufactured, or synthetic fibers. Suitable natural fibers may include cotton, jute, wool, flax, silk, cellulose, glass, and ceramic fibers. The fluid containment layer may include eco-fibers, such as bamboo fibers or eucalyptus fibers. Suitable manufactured fibers may include those formed from cellulose or protein. Suitable synthetic fibers may include polyester, polypropylene, polyethylene, Nylon, aramid, imide, acrylate fibers, or combination thereof. The fluid containment layer material may comprise polyester fibers, such as polybutylene terephthalate (PBT), polyethylene terephthalate (PET), polytrimethylene terephthalate (PTT), and co-polyester/polyester (CoPET/PET) adhesive bi-component fibers. The fibers may include polyacrylonitrile (PAN), oxidized polyacrylonitrile (Ox-PAN, OPAN, or PANOX), olefin, polyamide, polyetherketone (PEK), polyetheretherketone (PEEK), polyethersulfone (PES), or other polymeric fibers. The fibers may be selected for their melting and/or softening temperatures. The fibers may include mineral or ceramic fibers. The fibers may be or may include elastomeric fibers. Elastomeric fibers may provide cushioning performance and/or compressibility and recovery properties. Exemplary elastomeric fibers include elastic bicomponent PET, PBT, PTT, or a combination thereof. The fibers may be formed of any material that is capable of being carded and lapped into a three-dimensional structure. The fibers may be 100% virgin fibers or may contain fibers regenerated from postconsumer waste (for example, up to about 90% fibers regenerated from postconsumer waste or even up to 100% fibers regenerated from postconsumer waste). The fibers may have or may provide improved moisture absorption or moisture resistance characteristics, or both.

The fibers may be 100% virgin fibers or less. The fibers may include fibers regenerated from postconsumer waste (for example, up to about 90% fibers regenerated from postconsumer waste or even up to 100% fibers regenerated from postconsumer waste). The fibers may have geometries that are non-circular or non-cylindrical. The fluid containment layer may include or contain engineered aerogel structures. The fluid containment layer may include or be enriched with pyrolyzed organic bamboo additives.

The fibers, or at least a portion of the fibers, making up one or more layers of the material may include a hydrophilic finish or coating. The hydrophilic finish or coating may provide or enhance the ability of the fluid to distribute within the fluid containment layer, move through the fluid containment layer, transfer to an adjacent layer, or a combination thereof.

The fluid containment layer may include superabsorbent fibers. Superabsorbent fibers may absorb liquid or gel rapidly with excellent retention levels. Superabsorbent fibers may allow for absorption of fluid (e.g., liquid or gel) many times its own weight, which may allow for the fluid containment layer to absorb and hold liquid or gel many times its own weight. The superabsorbent fibers may be formed of a cellulose material or a synthetic polymeric material, for example. The superabsorbent fibers may be formed of a polyester, such as PET. The use of superabsorbent fibers may allow for the fluid containment layer to be filled with more gel or liquid than a traditional disinfection pad, which may extend the useful life of the disinfection pad. The use of superabsorbent fibers may also allow the fluid containment layer to be refilled with liquid or gel, thereby creating less waste and reducing the need for replacement of layers or of the entire disinfection pad. The superabsorbent fibers may be in a blend with other fibers. The superabsorbent fibers may be present in an amount of about 90% of the blend by weight or less, about 75% by weight or less, or about 60% by weight or less. The superabsorbent fibers may be present in an amount greater than 0%, about 1% by weight or greater, or about 5% by weight or greater.

The fluid containment layer (or any other layer of the material) may include a plurality of bi-component fibers. The bi-component fibers may be a thermoplastic lower melt bi-component fiber. The bi-component fibers may have a lower melting temperature than the other fibers within the mixture (e.g., a lower melting temperature than common or staple fibers). The bi-component fibers may be air laid or mechanically carded, lapped, and fused in space as a network so that the layered material may have structure and body and can be handled, laminated, fabricated, installed as a cut or molded part, or the like to provide desired properties. The bi-component fibers may include a core material and a sheath material around the core material. The sheath material may have a lower melting point than the core material. The web of fibrous material may be formed, at least in part, by heating the material to a temperature to soften the sheath material of at least some of the bi-component fibers.

The fluid containment layer (or any other layer of the layered material) may include a binder or binder fibers. Binder may be present in the fluid containment layer in an amount of about 100 percent by weight or less, about 80 percent by weight or less, about 60 percent by weight or less, about 50 percent by weight or less, about 40 percent by weight or less, about 30 percent by weight or less, about 25 percent by weight or less, or about 15 percent by weight or less. The fluid containment layer may be substantially free of binder. The fluid containment layer may be entirely free of binder. While referred to herein as fibers, it is also contemplated that the binder could be generally powder-like, spherical, or any shape capable of being received within interstitial spaces between other fibers and capable of binding the fluid containment layer together. The binder may have a softening and/or melting temperature of about 70° C. or greater, about 100° C. or greater, about 110° C. or greater, about 130° C. or greater, 180° C. or greater, about 200° C. or greater, about 225° C. or greater, about 230° C. or greater, or even about 250° C. or greater. For example, the binder may have a softening and/or melting temperature between about 70° C. and about 250° C. (with any range therein being contemplated). The fibers may be high-temperature thermoplastic materials. The fibers may include one or more of polyamideimide (PAI); high-performance polyamide (HPPA), such as Nylons; polyimide (PI); polyketone; polysulfone derivatives; polycyclohexane dimethyl-terephthalate (PCT); fluoropolymers; polyetherimide (PEI); polybenzimidazole (PBI); polyethylene terephthalate (PET); polybutylene terephthalate (PBT); polyphenylene sulfide; syndiotactic polystyrene; polyetherether ketone (PEEK); polyphenylene sulfide (PPS), polyether imide (PEI); and the like. The fluid containment layer may include polyacrylate and/or epoxy (e.g., thermoset and/or thermoplastic type) fibers. The fluid containment layer may include a multi-binder system. The fluid containment layer may include one or more elastomeric fiber materials acting as a binder. The fluid containment layer may include one or more sacrificial binder materials and/or binder materials having a lower melting temperature than other fibers within the layer.

Gel or liquid may be added to the fluid containment layer. The gel or liquid (referred to herein as a fluid) may be any product found to kill germs or particular bacteria, viruses, or other pathogens. The fluid may be poured over the fluid containment layer. The fluid containment layer may be at least partially submerged in the fluid. The fluid may be poured or otherwise deposited within the confines of the backing layer. The fluid containment layer may then be positioned within the backing layer. The fluid may be added a single time, and the disinfection pad may be discarded upon use or evaporation or drying of the fluid. The disinfection pad may be refillable so the fluid containment layer may be reused. The fluid containment layer may be saturated with the gel or liquid. The fluid containment layer may be filled beyond the saturation point (e.g., so fluid is retained within the backing layer for further absorption by the fluid containment layer upon release of fluid upon use of the disinfection pad).

The fluid containment layer may be impregnated with one or more powders. The powders may act to assist in filling the fluid containment layer with liquid or gel. The powder may be impregnated into the pad, and a liquid may be poured or otherwise added to the fluid containment layer. The addition of liquid to the impregnated powder may create a gel. The liquid may include alcohol (e.g., ethanol, isopropyl alcohol). This may provide faster processing, time saving, and/or cost saving to provide the liquid or gel disinfection fluid into the fluid containment layer.

The fibers of the fluid containment layer may have particles embedded therein. The particles may be embedded through an extrusion process. The particles present in the fibers may increase the surface area of the fiber by 50% or more, about 100% or more, by 200% or more, or by 500% or more as compared with a fiber that is free of embedded particles. The particles may increase the surface area of the fiber by about 1200% or less, about 1000% or less, or about 900% or less. The high surface area of the fiber may provide high adsorption properties. The particles may assist in removing or driving fluid through and/or across the fluid containment layer. Embedded particles may include, but are not limited to, wood, shells (e.g., fruit and/or nut shells, such as coconut shells or fibers thereon, hazelnut shells), activated carbon, sand (e.g., volcanic sand), or a combination thereof. For example, the fiber may be a PET fiber extruded with active carbon and/or volcanic sand.

The fibers and binders discussed herein in the context of the fluid containment layers may also be used to form any other layer of the layered material.

The weight of the fluid containment layer may depend upon the number of individual layers and/or thickness of the layers. The weight of the material may be impacted by the properties desired of the material, the conditions where the material will be used, or both. Certain applications may require a denser material. The weight may be generally variable throughout the material. The variation may occur, for example, as a result of one or more shaping procedures, one or more thinned areas, one or more areas having a density gradient, or a combination thereof.

The fluid containment layer itself may have a weight of about 400 g/m2 or greater, about 450 g/m2 or greater, or about 550 g/m2 or greater. The fluid containment layer itself may have a weight of about 800 g/m2 or less, about 750 g/m2 or less, or about 700 g/m2 or less. The weight may be generally uniform throughout the material. The weight may be generally variable throughout the material. The variation may occur, for example, as a result of one or more shaping procedures, one or more thinned areas, one or more areas having a density gradient, or a combination thereof.

The thickness of the fluid containment layer may depend upon the desired properties of the fluid containment layer, the desired properties of the disinfection pad as a whole, the interaction between layers of the material, or a combination thereof. The thickness (e.g., thickness at a particular point, maximum thickness, or average thickness) of the fluid containment layer may be about 0.5 mm or more, about 1 mm or more, or about 1.5 mm or more. The thickness (e.g., thickness at a particular point, maximum thickness, or average thickness) of the fluid containment layer may be about 40 mm or less, about 30 mm or less, about 25 mm or less, or about 15 mm or less.

As a non-limiting example, the fluid containment layer may have a weight between about 450 g/m2 and 700 g/m2 and a thickness of about 10 mm.

The fluid containment layer may have a generally uniform thickness. The fluid containment layer may have a generally uniform thickness prior to any shaping steps (e.g., molding or thermoforming). The fluid containment layer may have a variable thickness. The fluid containment layer may have a variable thickness as a result of one or more shaping procedures, one or more thinned areas, one or more compressed areas, or the like. The edges of the fluid containment layer (or of the disinfection pad) may be compressed, thereby creating a lower thickness at or around at least a portion of the edge of the fluid containment layer or disinfection pad.

The fibers forming the fluid containment layer, or one or more fibrous layers thereof, may be formed into a nonwoven web using nonwoven processes including, for example, blending fibers, carding, lapping, air laying, mechanical formation, or a combination thereof. Through these processes, the fibers may be oriented in a generally vertical direction or near-vertical direction (e.g., in a direction generally perpendicular to the longitudinal axis of the fluid containment layer). For example, the fluid containment layer or one or more fibrous layers thereof may include a carded and lapped material. When carded, the fibers may be arranged generally in the machine direction. When lapped, the fibers may be arranged to generally follow a generally sinusoidal shape. The fibers may be generally vertical (e.g., extending between surfaces in the thickness direction) between loops of the lapped structure. The fibers may be generally curved at the looped portions. The fibers may be opened and blended using conventional processes. The resulting structure formed may be a lofted fluid containment layer. The lofted fluid containment layer may be engineered for optimum weight, thickness, physical attributes, thermal conductivity, insulation properties, moisture absorption, or a combination thereof.

The fluid containment layer or one or more fibrous layers thereof may be formed, at least in part, through a carding process. The carding process may separate tufts of material into individual fibers. During the carding process, the fibers may be aligned in substantially parallel orientation with each other. A carding machine may be used to produce the web.

A carded web may undergo a lapping process to produce the fluid containment layer or fibrous layers within the fluid containment layer. The carded web may be rotary lapped, cross-lapped or vertically lapped, to form a voluminous or lofted nonwoven material. The carded web may be vertically lapped according to processes such as “Struto” or “V-Lap”, for example. This construction provides a web with relatively high structural integrity in the direction of the thickness of the fibrous layers, thereby minimizing the probability of the web falling apart during application, or in use, and/or providing compression resistance to the layered material. Carding and lapping processes may create nonwoven fibrous layers that have good compression resistance through the vertical cross-section (e.g., through the thickness of the layered material) and may enable the production of lower mass fibrous layers, especially with lofting to a higher thickness without adding significant amounts of fiber to the matrix. It is contemplated that a small amount of hollow conjugate fiber (i.e., in a small percentage) may improve lofting capability and resiliency to improve moisture absorption, physical integrity, or both. Such an arrangement also provides the ability to achieve a low density web with a relatively low bulk density.

The lapping process may create a looped, sinusoidal, or undulated appearance of the fibers when viewed from its cross-section prior to any compression operation. The loops may have generally curved or rounded portions (e.g., as opposed to sharp creases from a traditional pleating operation). The frequency of the loops or undulations may be varied during the lapping process. For example, having an increase in loops or undulations per area may increase the density and/or stiffness of the layer or layers of the material. Reducing the loops or undulations per area may increase the flexibility of the layer or layers and/or may decrease the density. The ability to vary the loop or undulation frequency during the lapping process may allow for properties of the material to be varied or controlled. It is contemplated that the loop or undulation frequency may be varied throughout the material. During the lapping process, the loop frequency may be dynamically controlled and/or adjusted. The adjustment may be made during the lapping of a layer of the material. For example, certain portions of the layer may have an increased frequency, while other portions of the layer or layers may have a frequency that is lower. The adjustment may be made during the lapping of different layers of the material. Different layers may be made to have different properties with different pleat frequencies. For example, one layer may have a loop frequency that is greater than or less than another layer of the layered material.

In an exemplary fluid containment layer or one or more fibrous layers thereof, the carded web, with the fibers generally extending in the machine direction, may then undergo a lapping process, creating a series of loops or undulations (e.g., appearing as peaks and valleys when viewed from the side or a cross section). The loops (e.g., line extending across an entire peak or valley) may extend across the surface of the material generally perpendicularly to the longitudinal axis of the fibrous layers, generally perpendicularly to the machine direction, or both.

As an example, the loops of the fluid containment layer may run generally parallel to the longitudinal axis of the item to which it is secured (e.g., a door). If the fluid containment layer is held or situated upright, the loops of the fluid containment layer may run generally perpendicular to the floor.

In another example, the loops of the fluid containment layer may run generally perpendicular to the longitudinal axis of the item to which it is secured (e.g., a door). If the fluid containment layer is held or situated upright, the loops of the fluid containment layer may run generally parallel to the floor.

The fluid containment layer or fibrous layers thereof may be formed by an air laying process. This air laying process may be employed instead of carding and/or lapping. In an air laying process, fibers are dispersed into a fast-moving air stream, and the fibers are then deposited from a suspended state onto a perforated screen to form a web. The deposition of the fibers may be performed by means of pressure or vacuum, for example. An air laid or mechanically formed web may be produced. The web may then be thermally bonded, air bonded, mechanically consolidated, the like, or combination thereof, to form a cohesive nonwoven fibrous layer. While air laying processes may provide a generally random orientation of fibers, there may be some fibers having an orientation that is generally in the vertical direction (e.g., vertical in the thickness direction) so that resiliency in the thickness direction of the material may be achieved.

During processing of the material, the fibrous layers may be compressed. Compression may occur during lamination, thermoforming in-situ, or the like. Compression may reduce thickness of the fibrous layers. The thickness may be reduced by 10% or more, about 25% or more, about 40% or more, or about 50% or more. The thickness may be reduced by about 80% or less, about 75% or less, about 67% or less, or about 60% or less. Upon compression, instead of a generally sinusoidal cross-section with generally straight segments between opposing loops, the segments between the loops may be generally C-shaped, S-shaped, Z-shaped, or otherwise curved, folded, or bent.

The layered material may include one or more intermediate layers, which may function to draw moisture from the fluid containment layer, from a layer directly adjacent, or both. The intermediate layer may be attached to one side of the fluid containment layer. The intermediate layer may be located between a contact layer and the fluid containment layer. The intermediate layer may be a wicking layer. The intermediate layer may be formed using any of the fibers and/or binders discussed herein with respect to the fluid containment layer. One or more acquisition layers may be made from Lycra, polyester, polyethylene terephthalate, or a combination thereof.

An intermediate layer may include one or more moisture transport layers, which may serve to transport the fluid held within the fluid containment layer to the contact layer. The intermediate layer may act to control the amount of fluid released upon pressure applied to the contact layer. The intermediate layer may draw fluid from the fluid containment layer and distribute the fluid over a wider surface area to reduce the amount of fluid released at the point of contact with the contact layer.

The disinfection pad may include a contact layer adapted to be contacted by a user. The contact layer may be the outermost layer of the disinfection pad. The contact layer may have a surface with a plurality of openings for release of the disinfection fluid upon pressure being applied to the contact layer. The contact layer may be formed of a material that is inherently porous. The contact layer may include a plurality of slits, holes, or other openings. The openings may be widened upon applying pressure to the contact layer. For example, the openings may be in a generally closed position when the contact layer is free of any pressure. Upon application of pressure from a finger or hand, for example, the openings may be pushed open to allow for the disinfection fluid (e.g., liquid or gel) to be released at the point of contact. The contact layer may act to retain the fluid within the disinfection pad prior to a pressure being applied. The contact layer may be formed of a flexible material. The contact layer may be formed of an elastic or elastomeric material. The contact layer may be formed of a polymeric material.

The contact layer may, upon application of pressure (e.g., from a hand or finger of a user), be pushed toward the layers beneath the contact layer. This may cause depression of the intermediate layer, the fluid containment layer, or both. The resilience of the fluid containment layer, which may be at least in part due to the orientation of the fibers, may cause the fluid containment layer, intermediate layer, and/or contact layer to return to its original position upon removal of the pressure. Therefore, the disinfection pad may be able to retain its shape, even upon repeated use or contact with the disinfection pad. The disinfection pad or layers thereof, such as the fluid containment layer, may provide cushioning. The disinfection pad or layers thereof, such as the fluid containment layer, may exhibit resilience. Resilience may be at least in part due to the orientation of the fibers, geometry of the fibers, denier of the fibers, composition of the fibers, the like, or a combination thereof. Resilience may be measured using a standardized compression force deflection or indentation force deflection test (e.g., ASTM D3574). The desired resilience may depend upon the application within which the layered material is used. The layered material may have a resilience suitable for its intended purpose.

The disinfection pad may include one or more additional layers. These additional layers may be or may include one or more moisture wicking layers. The one or more wicking layers may be located within the layered material. The wicking layers may be formed from a nonwoven material, a woven material, a knit material, a meltblown material (e.g., of thermoplastic polyurethane), or the like. One or more wicking layers may be made from Lycra, polyester, polyethylene terephthalate, or a combination thereof. The material may include one or more outer layers on an opposing surface of the fluid containment layer. The outer layer may be a moisture wicking layer. One or more of the layers, or the entire disinfection pad itself, may be flexible, stretchable, breathable, or a combination thereof. One or more of the layers may be a non-wicking material formed by any of the fibers and/or binders discussed herein with respect to the fluid containment. One or more of the wicking layers may be substituted by a non-wicking layer, such as a scrim, facing, mesh, or other permeable material.

A skin layer may be formed by melting a portion of one or more layers by applying heat in such a way that only a portion of the layer, such as the top surface, melts and then hardens to form a generally smooth surface. A scrim may be applied or secured to one or more of the layers. The disinfection pad may include a plurality of layers, some or all of which serve different functions or provide different properties to the layered material. The ability to combine layers having different properties may allow the disinfection pad to be customized based on the application. The layers may be combined so that the layered material provides cushioning with high resilience.

One or more layers of the disinfection pad may be or may form a thermoformable material. The disinfection pad may be or may include a layered material that may be formed with a broad range of densities and thicknesses and that contains a thermoplastic and/or thermoset binder. The thermoformable material may be heated and thermoformed into a specifically shaped thermoformed product.

A coating may be applied to form one or more layers of the disinfection pad. The coating may improve one or more characteristics of the disinfection pad. Oleophobic and/or hydrophobic treatments may be added. Flame retardants may be added. The coating may be anti-microbial, anti-fungal, have high infrared reflectance, moisture resistant, mildew resistant, or a combination thereof. Silver powder or other antimicrobial nano-powders can be added into one or more of the layers.

One of more of the layers of the disinfection pad may have hydrophobic properties. One or more of the layers of the disinfection pad may have hydrophilic properties. Entire layers may be hydrophobic or hydrophilic. A layer may have both hydrophobic and hydrophilic properties. For example, a layer may be formed from a mixture of hydrophobic fibers and hydrophilic fibers. The interfaces between layers may include one hydrophobic layer or portion abutting a hydrophilic layer or portion. One layer may be hydrophilic. Adjacent layers may, for example, be hydrophobic. The hydrophobic layers or portions thereof may function to transfer moisture to another layer of the layered material. The hydrophilic layers or portions thereof may function to absorb moisture (e.g., from one or more hydrophobic layers or portions). Fibers within the layers may be hydrophobic. Fibers within the layers may be hydrophilic.

Fibers of one or more layers of the disinfection pad, or one or more layers of the disinfection pad, may exhibit antimicrobial properties. The fibers may be treated with an antimicrobial substance. For example, silver or copper may be used. Fibers may be coated with silver, copper, or a combination thereof. The antimicrobial substance may be otherwise deposited on the surface of the fibers (e.g., via sputtering, electrostatic deposition). The antimicrobial substance may be part of the fibers. For example, silver particles, copper particles, or both, may be within fibers of the one or more layers of the layered material.

The disinfection pad or one or more layers thereof (e.g., the fluid containment layer) may be formed to have a thickness and density selected according to the required physical, moisture absorption/resistance, and air permeability properties desired of the finished layers (and/or the disinfection pad as a whole). The layers of the disinfection pad may be any thickness depending on the application, location of installation, shape, fibers used, fiber geometry and/or orientation, lofting of the fibrous layers, or other factors. The density of the layers may depend, in part, on the specific gravity of any additives incorporated into the material comprising the layer (such as nonwoven material), and/or the proportion of the final material that the additives constitute. The layered material may have a varying density and/or thickness along one or more of its dimensions. Bulk density generally is a function of the specific gravity of the fibers and the porosity of the material produced from the fibers, which can be considered to represent the packing density of the fibers.

Moisture absorption, moisture resistance, or a combination thereof of the disinfection pad (and/or its layers) may be impacted by the shape of the disinfection pad. The disinfection pad, or one or more of its layers, may be generally flat. The disinfection pad, or one of its layers, may be supplied as a sheet. The disinfection pad or one or more of its layers may be supplied in a roll. One or more layers of the disinfection pad may be laminated together (e.g., to supply the layered material as a sheet or roll and/or prior to any additional shaping or molding step). Layers of the disinfection pad may be fabricated into cut-to-print two-dimensional flat parts depending on the desired application. The disinfection pad or layers thereof may be formed into any shape. For example, the disinfection pad or layers thereof may be molded (e.g., into a three-dimensional shape) to generally match a desired shape. The finished disinfection pad or layers thereof may be molded-to-print into a three-dimensional shape for a desired application.

One or more of the layers may be attached to another layer of the disinfection pad. One layer may be attached to an adjacent layer (e.g., directly adjacent layer). Any suitable method of attachment may be employed, including use of adhesives, laminating techniques, heat sealing, or the like. The attachment may be permanent. The attachment may be temporary or repositionable. For example, the contact layer may be peeled away or otherwise disconnected from the backing layer to allow for the fluid containment layer to be replaced or refilled with fluid.

One or more layers may be attached to each other by a laminating process. The laminated layers may then be supplied as a roll or a sheet of the laminated product, which may be cut to fit the desired area. One or more layers may be attached to each other prior to any additional shaping or molding steps. One or more layers may include a thermoplastic component (e.g., binder or fibers) that melts and bonds to an adjacent surface upon exposure to heat. One or more layers may be attached to each other with an adhesive layer. The adhesive layer may be an adhesive. The adhesive may be a powder or may be applied in strips, sheets, or as a liquid or paste. The adhesive layer may extend along a surface of the fibrous layers, the wicking layers, the surface layers, or a combination thereof, to substantially cover the surface. The adhesive layer may be applied to a portion of the surface of one or more of the layers. The adhesive layer may be applied in a pattern (e.g., dots of adhesive applied to the surface). The adhesive layer may be applied in a uniform thickness. The adhesive layer may have varying thickness. The adhesive layer may be a single layer (e.g., a single adhesive). The adhesive layer may be multiple layers (e.g., an adhesive layer and a thermoplastic fiber layer). The adhesive layer may be a single layer of blended materials (e.g., an adhesive and thermoplastic fibers are blended in a single layer).

The layers may be directly attached to each other via other processes, such as by sewing, entanglement of fibers between layers, sealing, or other methods. The edges of layers may be sewn together. One or more layers may be sealed at the edges. For example, the outer layers may be sealed at the edges to encapsulate the interior layers. The contact layer may be secured to the backing layer. The contact layer and backing layer may act to encapsulate the layers located therebetween (e.g., the fluid containment layer, an intermediate layer, or both). The layers may be heated and/or compressed to seal all of the layers together. A double die system may be used, where the central portion of each die is insulated so as not to burn or melt the body of the material, and the edges of the dies are heated and pinched together such that the edges are sealed and the body of the material remains lofted. For example, heated pinch edge sealing may bond the layers together. The thickness at this pinched edge may be about 3 mm or less, about 2 mm or less, or about 1 mm or less and greater than 0 mm. One or more layers or one or more edges may be ultrasonically sealed. The edge may then be trimmed or cut after heating, compressing, pinching, sealing, the like, or combination thereof.

The disinfection pad may be secured to any area by any typical method of attaching one item to another item. For example, the disinfection pad may be secured to a surface via an adhesive. The disinfection pad may include mounting hardware for mounting the disinfection pad to a surface. The disinfection pad may be mounted or secured, for example, by hook and loop fastener, nails, screws, pins, bolts, snaps, rivets, tongue and groove fasteners, or other fastener. The disinfection pad may snap onto a surface, be friction fit, slide into engagement with a connector or otherwise engage to be secured to the intended area for the intended purpose. The method of attachment may be temporary. The method of attachment may be permanent. The method of attachment may allow the disinfection pad to be removed, repositioned, replaced, or any combination thereof.

The material as described herein may include printing such as logos, instructions for use, advertisements, or the like. Fabric printing methods that do not plug pores of the fabric, such as sublimation printing, may be employed, so functionality of the material is not impacted.

While described herein as a layered material, it is contemplated that some portions of a disinfection pad may be free of certain layers. For example, all layers may not be coextensive. It is also contemplated that two or more layers may be coextensive. While referred to herein as “layers,” it is contemplated that this includes discrete layers or portions within one or more materials. For example, a two layer material may include two discrete layers or a single material having two different portions.

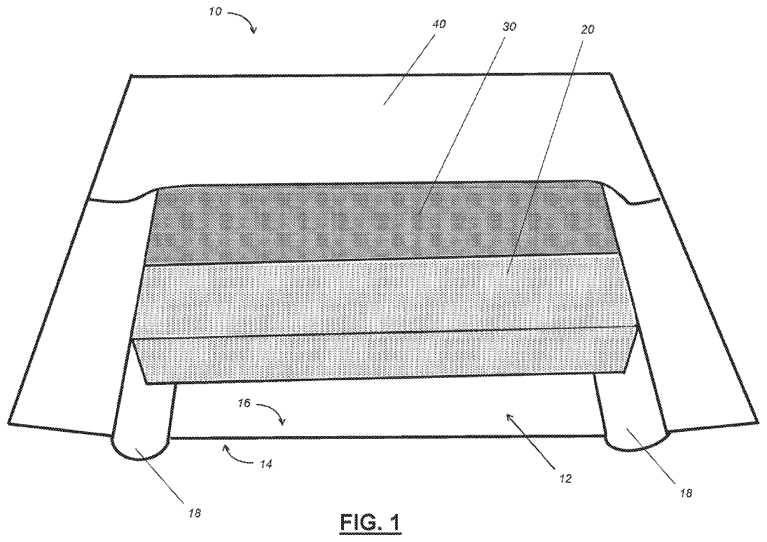

Turning now to the FIGURE, FIG. 1 illustrates a partial cutaway view of a disinfection pad 10 having multiple layers. A backing layer 12 has an outer side 14 that is adapted to face the surface upon which the disinfection pad is to be installed. The backing layer 12 has an internal side 16, which is adapted to face the other layers of the disinfection pad. The backing layer 12 includes optional channels 18, which contain additional fluid (e.g., disinfection or antibacterial liquid or gel).

The disinfection pad 10 includes a fluid containment layer 20, which is adapted to hold or contain the majority of the fluid within the disinfection pad. Fluid is permitted to travel or distribute throughout the fluid containment layer 20.

The disinfection pad 10 includes an intermediate layer 30 located between the fluid containment layer 20 and a contact layer 40. The intermediate layer 30 acts to control the amount of fluid dispensed at a point of pressure on the contact layer 40, distribute the fluid across a greater surface area, protect the containment layer, or a combination thereof.

The disinfection pad 10 includes a contact layer 40, which is an outermost layer of the disinfection pad. A user contacts the contact layer 40, and the pressure from the contact causes fluid to be released at least at the point of contact. The fluid acts to disinfect the point of contact, the part of the user that touched the disinfection pad, or both. The contact layer 40, therefore, is permeable (e.g., by a plurality of slits, openings, microvalves, or inherently permeable).

The contact layer 40 is attached at the edges to the backing layer 12, thereby encapsulating the fluid containment layer 20 and intermediate layer 30.

Any of the materials described herein may be combined with other materials described herein (e.g., in the same layer or in different layers of the layered material). The layers may be formed from different materials. Some layers, or all of the layers, may be formed from the same materials, or may include common materials or fibers. The type of materials forming the layers, order of the layers, number of layers, positioning of layers, thickness of layers, or a combination thereof, may be chosen based on the desired properties of each material, the desired weight, density and/or thickness of the material, the desired flexibility of the material (or locations of controlled flexibility), or a combination thereof. The layers may be selected to provide varying orientations of fibers.

While discussed in the context of a disinfection pad herein, the present teachings are not limited to such applications. The fluid containment layer as described herein may be useful in any application where liquid or gel is to be contained, absorbed, controllably released, or a combination thereof. While referred to herein as disinfecting liquid or gel, it is contemplated that other liquids or gels may be used, absorbed, or contained that do not necessarily have disinfecting properties.

Parts by weight as used herein refers to 100 parts by weight of the composition specifically referred to. Any numerical values recited in the above application include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01, or 0.1 as appropriate. These are only examples of what is specifically intended and all possible combinations of numerical values between the lowest value, and the highest value enumerated are to be expressly stated in this application in a similar manner. Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

### ELEMENT LIST

| | |
|---|---|
| 10 | Disinfection pad |
| 12 | Backing layer |
| 14 | Outer side |
| 16 | Internal side |
| 18 | Channels |
| 20 | Fluid containment layer |
| 30 | Intermediate layer |
| 40 | Contact layer |

1. An article comprising:
a fluid containment layer having a plurality of generally vertically oriented fibers when in an uncompressed state in the thickness direction;
wherein the fluid containment layer is adapted to absorb, hold, controllably release, or a combination thereof, a liquid or gel;
wherein the fluid containment layer is resilient and returns to its original shape upon release of pressure applied thereto;
wherein the fluid containment layer is suitable for use in a pad for controlled release of the liquid or gel; and
wherein the liquid or gel is a disinfecting liquid or gel.

2. (canceled)

3. The article of claim 1, wherein the pad is a self-disinfecting pad.

4. (canceled)

5. The article of claim 1, wherein the fluid containment layer maintains a generally even distribution of the liquid or gel within the fluid containment layer.

6. The article of claim 5, wherein upon release of the liquid or gel from the fluid containment layer at one or more points, remaining liquid or gel within the fluid containment layer disperses to maintain the generally even distribution.

7. The article of claim 1, wherein the fluid containment layer has a gradient density structure, wherein through the thickness, the density increases from one surface to an opposing surface.

8. The article of claim 1, wherein the fluid containment layer has a gradient structure where different portions of the fluid containment layer absorb or hold different amounts of fluid.

9. (canceled)

10. The article of claim 8, wherein the gradient structure is in the thickness direction.

11. The article of claim 8, wherein the fluid containment layer is a single layer having the gradient structure.

12. The article of claim 8, wherein the gradient structure is formed from two or more layers making up the fluid containment layer.

13. The article of claim 1, wherein the fluid containment layer allows for movement of the liquid or gel in multiple directions within the fluid containment layer to distribute the liquid or gel.

14. The article of claim 1, wherein the fluid containment layer is resistant to sag at its saturation point.

15. The article of claim 1, wherein the fluid containment layer comprises superabsorbent fibers.

16. The article of claim 1, wherein the fluid containment layer is impregnated with a powder so that upon addition of a liquid, a gel is created and held within the fluid containment layer.

17. The article of claim 1, wherein the fluid containment layer has a vertically lapped structure.

18. The article of claim 1, wherein the fluid containment layer is formed by an air laying process.

19. The article of claim 1, wherein the fluid containment layer is a shaped three-dimensional structure.

20. The article of claim 1, wherein the fluid containment layer or at least a portion thereof is generally planar.

21. The article of claim 19, wherein the fluid containment layer is thermoformable to allow the fluid containment layer to be formed into the shaped three-dimensional structure.

22. (canceled)

23. The article of claim 1, wherein the fluid containment layer is refillable.

24. A self-disinfecting pad including the article of claim 1.
